(19) United States
(12) Patent Application Publication
(10) Pub. No.: US 2015/0289836 A1
(43) Pub. Date: Oct. 15, 2015

(54) ULTRASONIC BLOOD PRESSURE MEASURING DEVICE AND ULTRASONIC BLOOD PRESSURE MEASURING METHOD

(71) Applicant: SEIKO EPSON CORPORATION, Tokyo (JP)

(72) Inventor: Hiromitsu MIZUKAMI, Shiojiri-shi (JP)

(21) Appl. No.: 14/683,587

(22) Filed: Apr. 10, 2015

(30) Foreign Application Priority Data
Apr. 11, 2014 (JP) 2014-081924
Aug. 5, 2014 (JP) 2014-159625

(51) Int. Cl. A61B 8/04; A61B 5/022; A61B 5/00; A61B 8/08; A61B 5/02

(52) U.S. Cl. CPC A61B 8/04; A61B 8/5223; A61B 5/02007; A61B 5/7246; A61B 5/022

(57) ABSTRACT

An ultrasonic blood pressure measuring device receives a reflected wave of an ultrasonic wave transmitted to a blood vessel, and measures the diameter of the blood vessel based on the reflected wave of the ultrasonic wave during at least one heartbeat period. Then, the ultrasonic blood pressure measuring device calculates cardiac systolic blood pressure and cardiac diastolic blood pressure from the maximum value of the blood vessel diameter, which appears after the peak of the degree of change in the measured blood vessel diameter, and the minimum value of the blood vessel diameter, which appears before the peak, using a correlation between the diameter of the blood vessel and blood pressure set in advance.

### CROSS REFERENCE

The entire disclosure of Japanese Patent Application No. 2014-081924, filed Apr. 11, 2014 and Japanese Patent Application No. 2014-159625, filed Aug. 5, 2014, are expressly incorporated by reference herein.

### BACKGROUND

1. Technical Field

The present invention relates to an ultrasonic blood pressure measuring device that measures blood pressure using an ultrasonic wave.

2. Related Art

A known method for measuring the blood pressure of a subject in a non-invasive manner uses a technique of measuring the diameter of the blood vessel of a subject using an ultrasonic wave and estimating the blood pressure from the blood vessel diameter. For example, JP-A-2004-41382 discloses a method of regarding the relationship between the blood vessel diameter and blood pressure as a non-linear function and calculating the blood pressure from the blood vessel diameter and a stiffness parameter β indicating the hardness of the blood vessel.

In general, in blood pressure measurement, the highest blood pressure (cardiac systolic blood pressure) and the lowest blood pressure (cardiac diastolic blood pressure) are measured. That is, when calculating the blood pressure using the relationship between the blood vessel diameter and blood pressure as described above, a cardiac systolic blood vessel diameter that is a blood vessel diameter at the time of the highest blood pressure (cardiac systolic blood pressure) and a cardiac diastolic blood vessel diameter that is a blood vessel diameter at the time of the lowest blood pressure (cardiac diastolic blood pressure) need to be measured first.

In a cuff type pressure sphygmomanometer used in known blood pressure measurement, there is a disadvantage in that about tens of seconds are required for measurement and continuous measurement is not possible. Therefore, quick and continuous blood pressure measurement has been demanded.

### SUMMARY

An advantage of some aspects of the invention is to enable quick and continuous measurement of the highest blood pressure (cardiac systolic blood pressure) and the lowest blood pressure (cardiac diastolic blood pressure) in blood pressure measurement using an ultrasonic wave.

A first aspect of the invention is directed to an ultrasonic blood pressure measuring device including: a transmission and reception unit that transmits an ultrasonic wave to a blood vessel and receives a reflected wave of the ultrasonic wave; a blood vessel diameter measuring unit that measures a diameter of the blood vessel based on the reflected wave of the ultrasonic wave during at least one heartbeat period; a peak calculation unit that calculates a peak of a degree of change in the blood vessel diameter; a blood vessel diameter acquisition unit that acquires a maximum value of the blood vessel diameter, which appears after the peak of the degree of change in the blood vessel diameter, as a cardiac systolic blood vessel diameter and acquires a minimum value of the blood vessel diameter, which appears before the peak, as a cardiac diastolic blood vessel diameter; and a blood pressure calculation unit that calculates cardiac systolic blood pressure and cardiac diastolic blood pressure from the cardiac systolic blood vessel diameter and the cardiac diastolic blood vessel diameter using a correlation between the diameter of the blood vessel and blood pressure set in advance.

As another aspect of the invention, the invention may be configured as an ultrasonic blood pressure measuring method including: receiving a reflected wave of an ultrasonic wave transmitted to a blood vessel and measuring a diameter of the blood vessel based on the reflected wave of the ultrasonic wave during at least one heartbeat period; calculating a peak of a degree of change in the blood vessel diameter; and calculating cardiac systolic blood pressure and cardiac diastolic blood pressure from a maximum value of the blood vessel diameter, which appears after the peak of the degree of change in the blood vessel diameter, and a minimum value of the blood vessel diameter, which appears before the peak, using a correlation between the diameter of the blood vessel and blood pressure set in advance.

According to the first aspect and the like of the invention, it is possible to measure the cardiac systolic blood pressure and the cardiac diastolic blood pressure for each heartbeat. The blood vessel repeats contraction and expansion due to the beating of the heart, and the blood vessel diameter changes so as to increase in cardiac systole and decrease in cardiac diastole. For example, the carotid artery has a feature that the blood vessel diameter increases rapidly in cardiac systole and decreases slowly in cardiac diastole. That is, since the degree of change in the blood vessel diameter in cardiac systole is large compared with that in cardiac diastole, the peak of the degree of change in the blood vessel diameter appears in cardiac systole. Accordingly, the minimum value of the blood vessel diameter appearing before the peak of the degree of change in the blood vessel diameter can be acquired as the cardiac diastolic blood vessel diameter, and the maximum value appearing after the peak can be acquired as the cardiac systolic blood vessel diameter. Therefore, since the cardiac systolic blood pressure and the cardiac diastolic blood pressure appearing for each heartbeat can be measured, it is possible to measure the highest blood pressure (cardiac systolic blood pressure) and the lowest blood pressure (cardiac diastolic blood pressure) quickly and continuously.

As a second aspect of the invention, the ultrasonic blood pressure measuring device according to the first aspect of the invention may be configured such that the blood vessel diameter acquisition unit acquires the cardiac systolic blood vessel diameter and the cardiac diastolic blood vessel diameter using a peak of a speed at which the blood vessel diameter changes as the peak of the degree of change.

According to the second aspect of the invention, a speed at which the blood vessel diameter changes is used as the degree of change in the blood vessel diameter.

As a third aspect of the invention, the ultrasonic blood pressure measuring device according to the first aspect of the invention may be configured such that the blood vessel diameter acquisition unit acquires the cardiac systolic blood vessel diameter and the cardiac diastolic blood vessel diameter using a peak of acceleration at which the blood vessel diameter changes as the peak of the degree of change.

According to the third aspect of the invention, acceleration at which the blood vessel diameter changes is used as the degree of change in the blood vessel diameter.

As a fourth aspect of the invention, the ultrasonic blood pressure measuring device according to any one of the first to third aspects of the invention may be configured such that the ultrasonic blood pressure measuring device further includes a correlation setting unit that sets the correlation.

According to the fourth aspect of the invention, a correlation between the blood vessel diameter and blood pressure is set. The correlation between the blood vessel diameter and blood pressure differs depending on an individual. Therefore, a calibration suitable for a subject can be realized by setting the correlation between the blood vessel diameter and blood pressure using the blood pressure measured separately, for example.

As a fifth aspect of the invention, the ultrasonic blood pressure measuring device according to any one of the first to fourth aspects of the invention may be configured such the blood vessel is an artery.

According to the fifth aspect of the invention, it is possible to measure the blood pressure of the artery.

As a sixth aspect of the invention, the ultrasonic blood pressure measuring device according to any one of the first to fifth aspects of the invention may be configured such that the ultrasonic blood pressure measuring device further includes a storage unit that stores the correlation as a look-up table of the blood vessel diameter and the blood pressure, and the blood pressure calculation unit calculates cardiac systolic blood pressure and cardiac diastolic blood pressure from the cardiac systolic blood vessel diameter and the cardiac diastolic blood vessel diameter with reference to the look-up table.

According to the sixth aspect of the invention, it is possible to store the correlation between the blood vessel diameter and blood pressure as a look-up table. Therefore, it is possible to reduce the calculation load when calculating the blood pressure.

### BRIEF DESCRIPTION OF THE DRAWINGS

The invention will be described with reference to the accompanying drawings, wherein like numbers reference like elements.

FIG. 1 is an application example of an ultrasonic blood pressure measuring device.

FIG. 2 is an explanatory diagram of the measurement of a blood vessel diameter using an ultrasonic wave.

FIG. 3 is a graph showing the relationship between the blood vessel diameter and blood pressure.

FIG. 4A is a graph of a blood vessel diameter variation waveform, and FIG. 4B is a graph of a variation acceleration waveform of the blood vessel diameter.

FIG. 5 is a diagram showing the functional configuration of the ultrasonic blood pressure measuring device.

FIG. 6 is a flowchart of the blood pressure measuring process.

FIG. 7A is a graph of a blood vessel diameter variation waveform, and FIG. 7B is a graph of a variation speed waveform of the blood vessel diameter.

FIG. 8 is a diagram showing a modification example of correlation setting data.

### DESCRIPTION OF EXEMPLARY EMBODIMENTS

### Overall Configuration

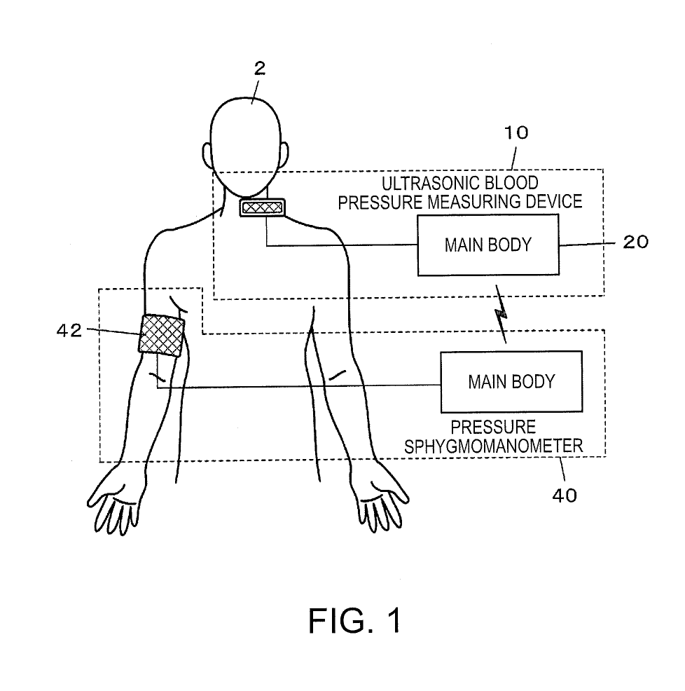

FIG. 1 is a diagram showing an application example of an ultrasonic blood pressure measuring device 10 in the present embodiment. The ultrasonic blood pressure measuring device 10 is a device that measures the blood pressure of a subject 2 in a non-pressure manner using an ultrasonic wave, and includes a main body 20 and an ultrasonic probe 30.

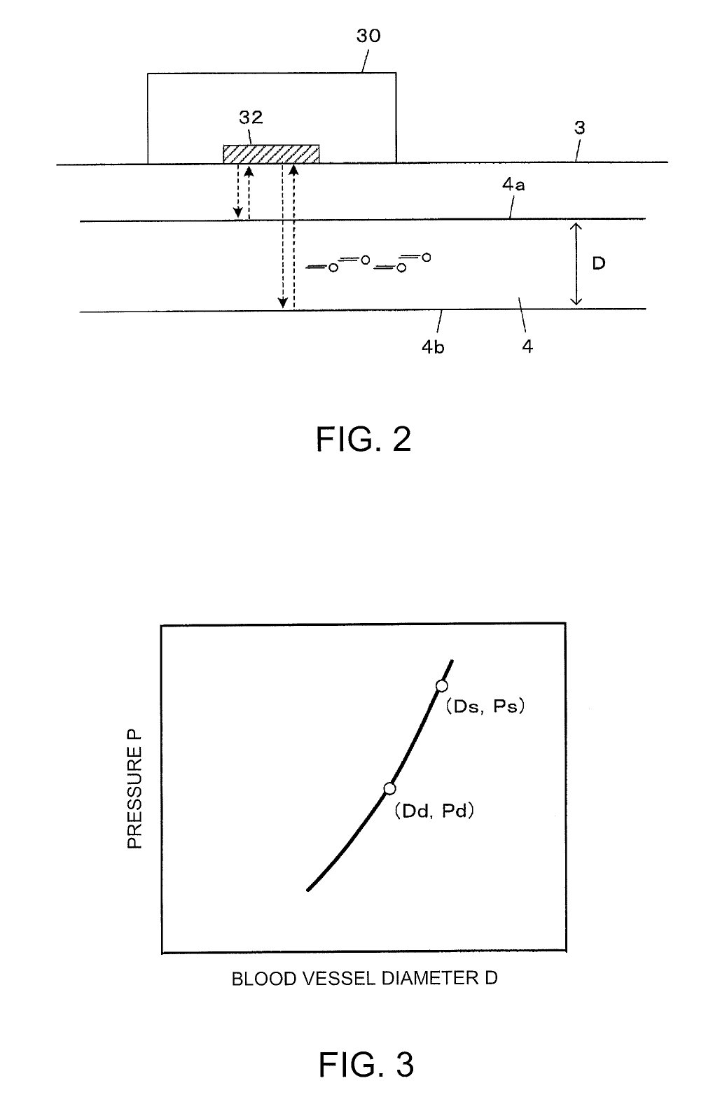

The ultrasonic probe 30 has an ultrasonic transducer 32 (refer to FIG. 2) to transmit and receive an ultrasonic pulse signal or burst signal of, for example, several to tens of MHz, and outputs a received signal to the main body 20. The ultrasonic probe 30 is attached to the neck skin surface of the subject 2 so that the ultrasonic transducer 32 is located immediately above the carotid artery of the subject 2, for example. Here, “immediately above” is used as an operating manual expression when operating the ultrasonic probe 30 for easy understanding. To be precise, “immediately above” refers to the positional relationship when the carotid artery is located on the straight line of irradiation of the ultrasonic wave emitted from the ultrasonic transducer 32.

The main body 20 is connected to the ultrasonic probe 30 by a cable, and measures the blood pressure of the subject 2 using the ultrasonic probe 30. Specifically, the main body 20 emits an ultrasonic wave toward the blood vessel (for example, carotid artery) of the subject 2 using the ultrasonic probe 30, measures a blood vessel diameter based on the received signal of the reflected wave, and calculates the blood pressure of the subject 2 based on the measured blood vessel diameter. In particular, the present embodiment is characterized in that cardiac systolic blood pressure (highest blood pressure) and cardiac diastolic blood pressure (lowest blood pressure) are measured and output for each heartbeat. In general, terms of systolic blood pressure and diastolic blood pressure are used. In the present embodiment, however, contraction and expansion of the blood vessel are also handled. Therefore, in order to avoid confusion, terms of cardiac systolic blood pressure and cardiac diastolic blood pressure are used.

In addition, in calculating the blood pressure based on the blood vessel diameter, it is necessary to measure the blood pressure for calibration apart from the blood vessel diameter. For this blood pressure measurement, it is assumed that a pressure sphygmomanometer 40 is used in the present embodiment. The pressure sphygmomanometer 40 measures the blood pressure of the arm artery of the subject 2 in a state where a cuff for pressure 42 is wound around the upper arm of the subject 2. After calibration, the cuff 42 is detached from the subject 2. Then, the blood pressure of the subject 2 is measured in a non-pressure manner, that is, in a non-invasive manner using the ultrasonic probe 30.

### Principle

### 1. Measurement of Blood Vessel Diameter

First, measurement of a blood vessel diameter using an ultrasonic wave will be described. FIG. 2 is a diagram for explaining the measurement of the blood vessel diameter using an ultrasonic wave, and shows a cross-sectional view in a long-axis direction of a blood vessel 4. As shown in FIG. 2, in measurement, the ultrasonic probe 30 is attached to the neck of the subject 2 so that the ultrasonic transducer 32 is in close contact with a skin surface 3 immediately above the blood vessel 4.

From the ultrasonic transducer 32, an ultrasonic wave is transmitted in a downward direction in FIG. 2. The ultrasonic wave has a characteristic of being largely reflected on the boundary surface of the medium. That is, when the blood vessel 4 is located immediately below the ultrasonic transducer 32, some of the ultrasonic waves transmitted from the ultrasonic transducer 32 are reflected on a front wall 4a and a rear wall 4b of the blood vessel 4, and strong reflected waves reflected from the front wall 4a and the rear wall 4b appear in the reflected wave signal in the ultrasonic transducer 32. From the time difference between the reflected wave from the front wall 4a and the reflected wave from the rear wall 4b and the propagation speed of the ultrasonic wave, a blood vessel diameter D is calculated. Since the propagation speed of the ultrasonic wave is known, it is possible to calculate the blood vessel diameter D if the time difference between reflected waves can be measured. The measurement of the blood vessel diameter using an ultrasonic wave is repeatedly performed at predetermined periods of several to tens of milliseconds, for example. The measurement unit is referred to as a “frame” hereinafter.

### 2. Correlation Between Blood Vessel Diameter and Blood Pressure

Next, measurement of blood pressure based on the blood vessel diameter will be described. FIG. 3 is a graph showing the correspondence relationship between the blood vessel diameter D and blood pressure P. As shown in FIG. 3, correlation between the blood vessel diameter D and the blood pressure P is a non-linear relationship, and it is known that the correlation between the blood vessel diameter D and the blood pressure P can be expressed by the correlation equation shown in the following equation (1).

P=Pd·exp[β(D/Dd−1)] (1)

Here, β=ln(Ps/Pd)/(Ds/Dd−1) (2)

In the equations (1) and (2), “Pd” is a cardiac diastolic blood pressure (lowest blood pressure), “Dd” is a cardiac diastolic blood vessel diameter that is a blood vessel diameter at the time of cardiac diastolic blood pressure, “Ps” is a cardiac systolic blood pressure (highest blood pressure), “Ds” is a cardiac systolic blood vessel diameter that is a blood vessel diameter at the time of cardiac systolic blood pressure, and “P” is a blood vessel elasticity index value called a stiffness parameter.

In order to calculate the blood pressure P from the blood vessel diameter D using the correlation equation (1), it is necessary to set the cardiac diastolic blood pressure Pd, the cardiac diastolic blood vessel diameter Dd, the cardiac systolic blood pressure Ps, the cardiac systolic blood vessel diameter Ds, and the stiffness parameter β that are constants of the equations (1) and (2) and to perform a calibration to define the correlation equation (1). Therefore, in the present embodiment, the cardiac diastolic blood vessel diameter Dd and the cardiac systolic blood vessel diameter Ds are measured using the ultrasonic probe 30 and the cardiac diastolic blood pressure Pd and the cardiac systolic blood pressure Ps are measured using the pressure sphygmomanometer 40, and the stiffness parameter β is calculated using the measurement values Dd, Ds, Pd, and Ps, thereby defining the correlation equation (1). In addition, the cardiac diastolic blood pressure Pd and the cardiac systolic blood pressure Ps for calibration do not need to be measured by the pressure sphygmomanometer 40, and may be measured by different measuring means.

After calibration, based on the measured blood vessel diameter D, it is possible to estimate the blood pressure P from the correlation equation (1). That is, in the present embodiment, a cardiac systolic blood vessel diameter Ds1 and a cardiac diastolic blood vessel diameter Dd1 are measured by ultrasonic measurement, and cardiac systolic blood pressure Ps1 and cardiac diastolic blood pressure Pd1 are calculated from the correlation equation (1) based on the cardiac systolic blood vessel diameter Ds1 and the cardiac diastolic blood vessel diameter Dd1 that have been measured.

### 3. Measurement of Cardiac Systolic Blood Vessel Diameter Ds1 and Cardiac Diastolic Blood Vessel Diameter Dd1

Next, measurement of the cardiac systolic blood vessel diameter Ds1 and the cardiac diastolic blood vessel diameter Dd1 using an ultrasonic wave will be described. A blood vessel repeats contraction and expansion approximately isotropically due to the beating of the heart. That is, the blood vessel diameter varies approximately similar to blood pressure.

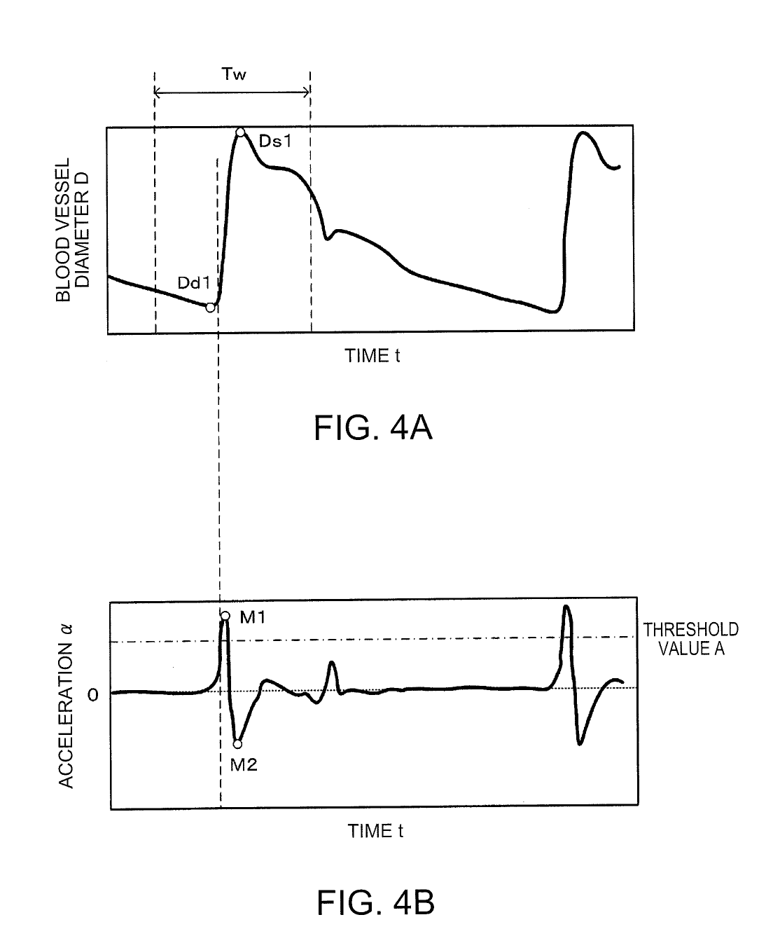

FIG. 4A is a blood vessel diameter variation waveform showing a temporal change in the blood vessel diameter of the carotid artery in one heartbeat period. In FIG. 4A, the horizontal axis indicates time t, and the vertical axis indicates the blood vessel diameter D. One heartbeat period includes diastole and systole. That is, the blood vessel diameter increases and accordingly the carotid artery vessel expands rapidly from cardiac systole according to the beating of the heart. In cardiac diastole, the blood vessel diameter decreases slowly to return to the original thickness. Thus, since the blood vessel diameter increases rapidly in cardiac systole, a blood vessel diameter variation waveform rises rapidly. However, since the blood vessel diameter decreases slowly in cardiac diastole, the blood vessel diameter variation waveform falls slowly. In the blood vessel diameter variation waveform, the minimum value of the blood vessel diameter is a cardiac diastolic blood vessel diameter Dd1, and the maximum value of the blood vessel diameter is a cardiac systolic blood vessel diameter Ds1. In the present embodiment, the cardiac systolic blood vessel diameter Ds1 and the cardiac diastolic blood vessel diameter Dd1 are acquired using the degree of change in the blood vessel diameter.

FIG. 4B is a waveform showing variation acceleration that is obtained from the blood vessel diameter variation waveform shown in FIG. 4A and is an example of the degree of change in the blood vessel diameter. The variation acceleration waveform of the blood vessel diameter is obtained by differentiating the blood vessel diameter waveform between adjacent frames twice in a time direction based on the frame rate.

Since the blood vessel diameter D decreases in cardiac diastole after increasing rapidly in cardiac systole, a positive peak (maximum value) M1 and a negative peak (minimum value) M2 appear in the cardiac systole in the variation acceleration waveform of the blood vessel diameter. Also in the cardiac diastole, a positive peak (maximum value) and a negative peak (minimum value) appear. However, since a change in the blood vessel diameter is gentle compared with that in the cardiac systole, the positive peak (maximum value) and the negative peak (minimum value) in the cardiac diastole are small peaks.

That is, the cardiac systolic blood vessel diameter Ds1 and the cardiac diastolic blood vessel diameter Dd1 appear before and after the positive peak M1 in cardiac systole of the variation acceleration waveform of the blood vessel diameter. Specifically, the peak M1 indicates a peak of the blood vessel diameter, the cardiac systolic blood vessel diameter Ds1 appears as a maximum value immediately after the peak M1, and the cardiac diastolic blood vessel diameter Dd1 appears as a minimum value immediately before the peak M1.

Therefore, a predetermined period Tw including the peak M1 of the variation acceleration waveform of the blood vessel diameter is set, and the maximum value of the blood vessel diameter D in the predetermined period Tw is detected as the cardiac systolic blood vessel diameter Ds1 and the minimum value of the blood vessel diameter D is detected as the cardiac diastolic blood vessel diameter Dd1. The predetermined period Tw is set so as to include the maximum value (cardiac systolic blood vessel diameter Ds1) and the minimum value (cardiac diastolic blood vessel diameter Dd1) of the blood vessel diameter variation waveform. Specifically, the predetermined period Tw is set as a period that is longer than the length of cardiac systole and is shorter than one heartbeat period, and is about hundreds of milliseconds, for example, “100 to 300 milliseconds”.

In addition, the peak M1 in the variation acceleration waveform of the blood vessel diameter is detected by the variation acceleration exceeding a predetermined threshold value A, and a time at which the peak M1 is detected is determined to be the peak. The predetermined threshold value A is set to a value that is smaller than the peak M1 of cardiac systole in the variation acceleration waveform and is larger than the peak of cardiac diastole. In addition, the variation acceleration waveform of the blood vessel diameter differs depending on the subject 2. Therefore, for example, it is also possible to acquire a variation acceleration waveform from a blood vessel diameter variation waveform acquired in advance at the time of calibration or the like and to set the threshold value A according to the magnitude of the positive peak in the variation acceleration waveform.

The cardiac systolic blood vessel diameter Ds1 and the cardiac diastolic blood vessel diameter Dd1 are calculated after the predetermined period Tw has ended. That is, after the predetermined period Tw has ended, the cardiac systolic blood vessel diameter Ds1 that is the maximum value of the blood vessel diameter in the predetermined period Tw and the cardiac diastolic blood vessel diameter Dd1 that is the minimum value of the blood vessel diameter are calculated, and the cardiac systolic blood pressure Ps1. and the cardiac diastolic blood pressure Pd1 are calculated from equation (1), thereby calculating the blood pressure. The time required for this processing is “about 100 to 200 milliseconds” at most. Accordingly, for each heartbeat, the cardiac systolic blood pressure Ps1 and the cardiac diastolic blood pressure Pd1 in the heartbeat can be immediately calculated and output before the next heartbeat arrives. As a result, it is possible to realize quick and continuous measurement.

### Functional Configuration

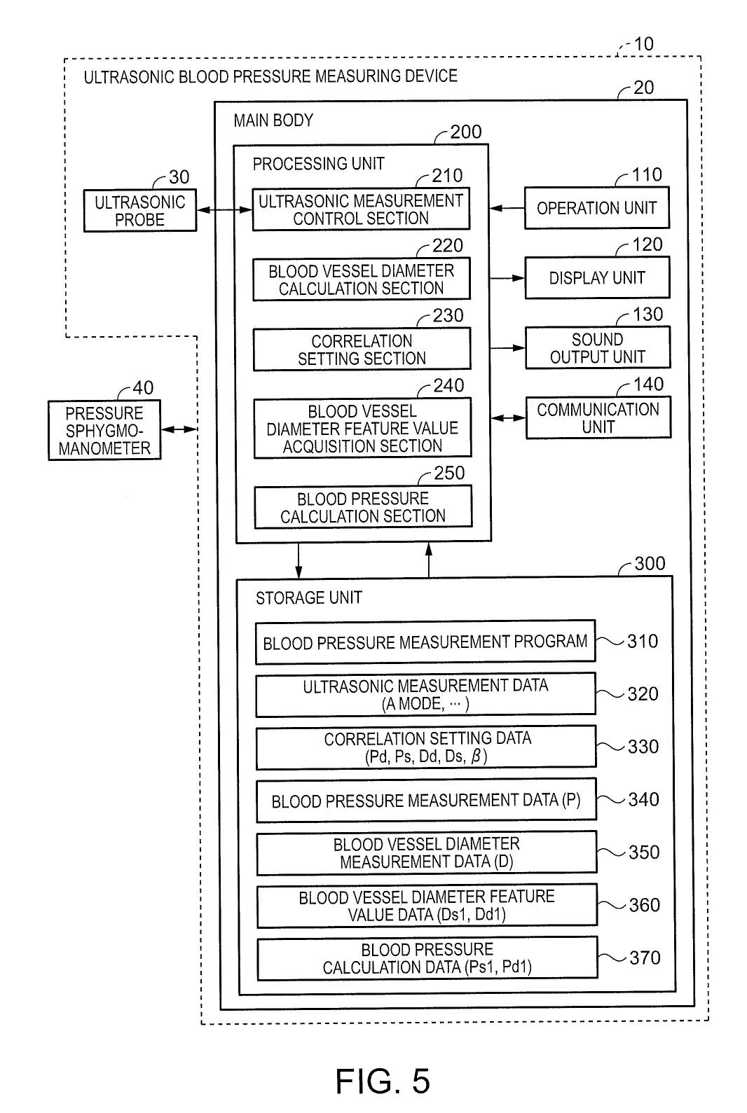

FIG. 5 is a diagram showing the functional configuration of the ultrasonic blood pressure measuring device 10. As shown in FIG. 5, the ultrasonic blood pressure measuring device 10 is configured to include the ultrasonic probe 30 and the main body 20. The main body 20 is configured to include an operation unit 110, a display unit 120, a sound output unit 130, a communication unit 140, a processing unit 200, and a storage unit 300.

The operation unit 110 is implemented by input devices, such as button switches, a touch panel, and various sensors, and outputs an operation signal corresponding to the operation to the processing unit 200. The display unit 120 is implemented by a display device, such as a liquid crystal display (LCD), and performs various kinds of display according to the display signal from the processing unit 200. The sound output unit 130 is implemented by a sound output device, such as a speaker, and outputs various sounds based on the sound signal from the processing unit 200. The communication unit 140 is implemented by a wireless communication device, such as a wireless local area network (LAN) or Bluetooth (registered trademark), and performs communication with an external device (mainly, the pressure sphygmomanometer 40).

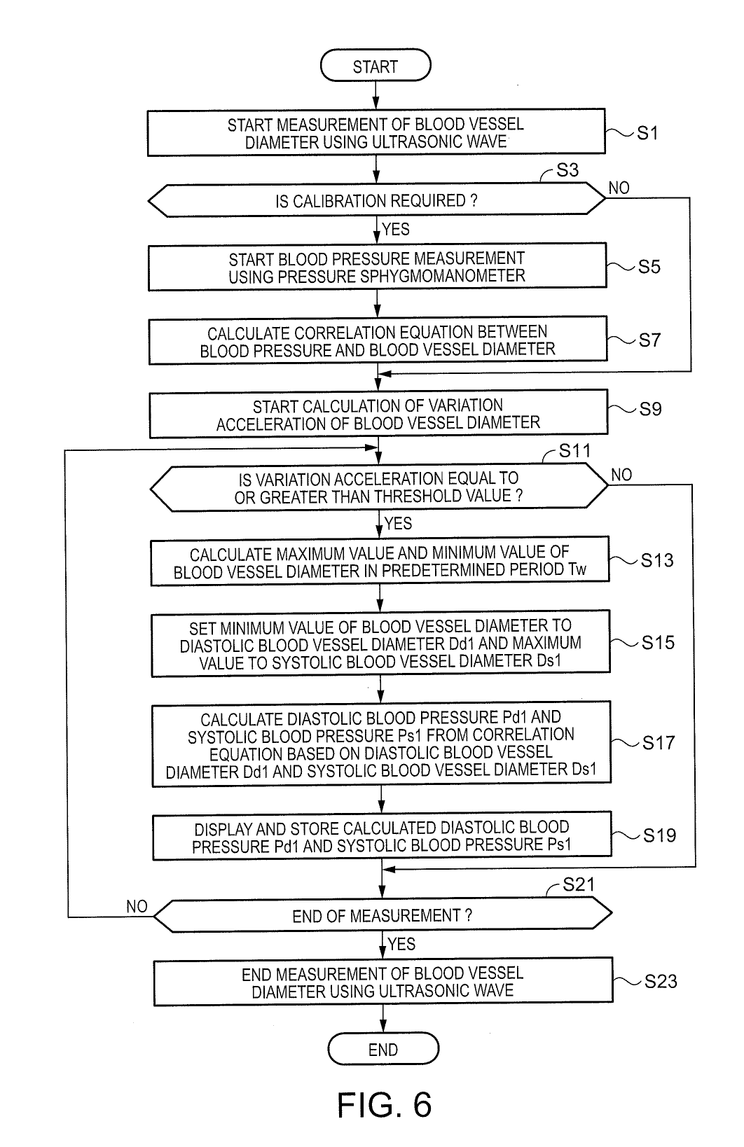

The processing unit 200 is implemented by a microprocessor, such as a central processing unit (CPU) or a digital signal processor (DSP), or an electronic component, such as an application specific integrated circuit (ASIC) or an integrated circuit (IC) memory. The processing unit 200 controls the operation of the ultrasonic blood pressure measuring device 10 by executing various kinds of arithmetic processing based on a program or data stored in the storage unit 300, an operation signal from the operation unit 110, or the like. In addition, the processing unit 200 includes an ultrasonic measurement control section 210, a blood vessel diameter calculation section 220, a correlation setting section 230, a blood vessel diameter feature value acquisition section 240, and a blood pressure calculation section 250, and executes blood pressure measurement processing (refer to FIG. 6) according to a blood pressure measurement program 310.

The ultrasonic measurement control section 210 controls the transmission and reception of an ultrasonic wave in the ultrasonic probe 30. Specifically, the ultrasonic measurement control section 210 transmits an ultrasonic wave from the ultrasonic probe 30 at the transmission timing of a predetermined period. In addition, the ultrasonic measurement control section 210 performs amplification or the like of the signal of the reflected wave of the ultrasonic wave received by the ultrasonic probe 30. Based on the received signal of the reflected wave of the ultrasonic wave received by the ultrasonic probe 30, ultrasonic measurement data 320 of each mode, such as an A mode, a B mode, or an M mode, is generated.

The blood vessel diameter calculation section 220 calculates a blood vessel diameter based on the received signal of the reflected wave of the ultrasonic wave received by the ultrasonic probe 30. That is, the reception of the reflected wave from each of the front wall 4a and the rear wall 4b of the blood vessel 4 is determined from the signal strength of the received signal. Then, the blood vessel diameter is calculated using the time difference between the reception times of the respective reflected waves. Since the transmission of the ultrasonic wave and the reception of the reflected wave by the ultrasonic probe 30 are performed when necessary, the calculation of the blood vessel diameter is repeatedly executed at every predetermined time (for example, at time intervals that can be called almost real time, such as several to tens of milliseconds). As a result, a waveform (refer to FIG. 4A) showing the variation of the blood vessel diameter is obtained. The blood vessel diameter obtained by the blood vessel diameter calculation section 220 is stored as blood vessel diameter measurement data 350 so as to match the measurement time.

The correlation setting section 230 sets the correlation between the blood vessel diameter D and the blood pressure P based on the calculation result of the blood vessel diameter calculation section 220 and the measurement result of the pressure sphygmomanometer 40. That is, the correlation equation (1) indicating the correlation between the blood vessel diameter and blood pressure is set and defined by calculating the stiffness parameter β given by equation (2) from the cardiac systolic blood pressure Ps, the cardiac diastolic blood pressure Pd, the cardiac systolic blood vessel diameter Ds, and the cardiac diastolic blood vessel diameter Diagnostic Dd. The correlation setting section 230 can also be said to be a calibration section that calibrates the correlation between the blood vessel diameter and blood pressure.

The cardiac systolic blood pressure Ps and the cardiac diastolic blood pressure Pd are measured by the pressure sphygmomanometer 40. Blood pressure measurement using the pressure sphygmomanometer 40 takes a time of about several to tens of seconds. The cardiac systolic blood vessel diameter Ds and the cardiac diastolic blood vessel diameter Dd are calculated from the blood vessel diameter calculated by the blood vessel diameter calculation section 220, the calculation by the blood vessel diameter calculation section 220 being performed in parallel with the blood pressure measurement using the pressure sphygmomanometer 40. That is, the maximum value and the minimum value of the blood vessel diameter are detected for each heartbeat, and the average value of the maximum values is set to the cardiac systolic blood vessel diameter Ds and the average value of the minimum values is set to the cardiac diastolic blood vessel diameter Dd.

The blood pressure measured by the pressure sphygmomanometer 40 is stored as blood pressure measurement data 340 so as to match the measurement time. In addition, the correlation between the blood vessel diameter and blood pressure set by the correlation setting section 230 is stored as correlation setting data 330. Specifically, the correlation setting data 330 stores the values of the parameter Ds, Dd, Ps, Pd, and β defining the correlation equation (1).

The blood vessel diameter feature value acquisition section 240 calculates the cardiac systolic blood vessel diameter Ds1 and the cardiac diastolic blood vessel diameter Dd1, which are feature values of the blood vessel diameter, from the blood vessel diameter calculated by the blood vessel diameter calculation section 220. That is, the blood vessel diameter feature value acquisition section 240 calculates the variation acceleration of the blood vessel diameter from the temporal change in the blood vessel diameter, regards variation acceleration when the variation acceleration exceeds a predetermined threshold value as the positive peak M1 (value of the peak: maximum value), and sets the predetermined period Tw with the time of the peak M1 as a reference. Then, the maximum value and the minimum value of the blood vessel diameter in the set predetermined period Tw are determined, and the determined maximum value is set to the cardiac systolic blood vessel diameter Ds1 and the minimum value is set to the cardiac diastolic blood vessel diameter Dd1. The blood vessel diameter feature value acquisition section 240 corresponds to a peak calculation section and a blood vessel diameter acquisition section. The feature values (cardiac systolic blood vessel diameter Ds1 and cardiac diastolic blood vessel diameter Dd1) of the blood vessel diameter calculated by the blood vessel diameter feature value acquisition section 240 are stored as blood vessel diameter feature value data 360.

The blood pressure calculation section 250 calculates the cardiac systolic blood pressure Ps1 and the cardiac diastolic blood pressure Pd1, according to the correlation equation (1) set by the correlation setting section 230, based on the cardiac systolic blood vessel diameter Ds1 and the cardiac diastolic blood vessel diameter Dd1 that are feature values of the blood vessel diameter calculated by the blood vessel diameter feature value acquisition section 240. The systolic blood pressure Ps1 and the cardiac diastolic blood pressure Pd1 calculated by the blood pressure calculation section 250 are stored as blood pressure calculation data 370 so as to match the measurement time.

The storage unit 300 is implemented by a storage device, such as a read only memory (ROM), a random access memory (RAM), or a hard disk. The storage unit 300 stores a program or data required for the processing unit 200 to perform overall control of the ultrasonic blood pressure measuring device 10, and is used as a working area of the processing unit 200. In addition, the calculation results of the processing unit 200, operation data from the operation unit 110, and the like are temporarily stored in the storage unit 300. In the present embodiment, the blood pressure measurement program 310, the ultrasonic measurement data 320, the correlation setting data 330, the blood pressure measurement data 340, the blood vessel diameter measurement data 350, the blood vessel diameter feature value data 360, and the blood pressure calculation data 370 are stored in the storage unit 300.

### Process Flow

FIG. 6 is a flowchart illustrating the flow of the blood pressure measuring process. This process is realized by the processing unit 200 that executes the blood pressure measurement program 310.

Referring to FIG. 6, first, the measurement of a blood vessel diameter using an ultrasonic wave is started when the ultrasonic measurement control section 210 starts control to transmit and receive an ultrasonic wave to and from the ultrasonic probe 30 and the blood vessel diameter calculation section 220 starts the measurement of the blood vessel diameter based on the received signal of the reflected wave of the ultrasonic wave (step S1).

Then, the processing unit 200 determines whether or not calibration is required. For example, the processing unit 200 determines that calibration is required when the subject 2 performs blood pressure measurement with the device for the first time or when a predetermined amount of time has passed from the previous measurement. If calibration is required (step S3: YES), for example, a message is displayed on the display unit 120 to give an instruction to attach the cuff 42 to the subject 2 and measure the blood pressure with the pressure sphygmomanometer 40. Accordingly, blood pressure measurement of the subject 2 using the pressure sphygmomanometer 40 is started (step S5). When the blood pressure measurement using the pressure sphygmomanometer 40 ends and the cardiac systolic blood pressure (cardiac systolic blood pressure) Ps and the cardiac diastolic blood pressure (cardiac diastolic blood pressure) Pd are measured, the correlation setting section 230 calculates the stiffness parameter β from the blood vessel diameter obtained by ultrasonic measurement and the blood pressure measured by the pressure sphygmomanometer 40, and calculates the correlation equation (1) between the blood vessel diameter and the blood pressure (step S7). This is the calibration.

After the calibration ends, the blood vessel diameter feature value acquisition section 240 starts the calculation of the variation acceleration of the blood vessel diameter by ultrasonic measurement (step S9). Then, the calculated variation acceleration is compared with the predetermined threshold value A. If the variation acceleration exceeds the predetermined threshold value A (step S11: YES), the predetermined period Tw including the time at which the variation acceleration exceeds the predetermined threshold value A is set, and the maximum value and the minimum value of the blood vessel diameter in the predetermined period Tw are calculated (step S13). Then, the maximum value is set to the cardiac systolic blood vessel diameter Ds1, and the minimum value is set to the cardiac diastolic blood vessel diameter Dd1 (step S15).

Then, the blood pressure calculation section 250 calculates the cardiac systolic blood pressure Ps1 and the cardiac diastolic blood pressure Pd1 from the correlation equation (1) based on the cardiac systolic blood vessel diameter Ds1 and the cardiac diastolic blood vessel diameter Dd1 that have been calculated (step S17). Then, the cardiac systolic blood pressure Ps1 and the cardiac diastolic blood pressure Pd1 that have been calculated are displayed on the display unit 120, and are stored as the blood pressure calculation data 370 (step S19).

Then, the processing unit 200 determines whether or not the blood pressure measurement using an ultrasonic wave has ended. If the blood pressure measurement has not ended (step S21: NO), the process returns to step S11 to perform blood pressure measurement for the next heartbeat period. On the other hand, if the blood pressure measurement has ended (step S21: YES), the ultrasonic measurement control section 210 ends the transmission and reception of the ultrasonic wave by the ultrasonic probe 30. After ending the measurement of the blood vessel diameter using an ultrasonic wave (step S23), this process is ended.

### Effects

Thus, according to the ultrasonic blood pressure measuring device 10 of the present embodiment, the cardiac systolic blood pressure (highest blood pressure) and the cardiac diastolic blood pressure (lowest blood pressure) can be measured for each heartbeat. That is, the variation acceleration of the blood vessel diameter measured using an ultrasonic wave is calculated, the peak M1 of the variation acceleration is detected by comparing the acceleration with the predetermined threshold value A, and the predetermined period Tw including the peak M1 as a reference is set. Then, the minimum value of the blood vessel diameter in the predetermined period Tw is set to the cardiac diastolic blood vessel diameter Dd1, and the maximum value of the blood vessel diameter is set to the cardiac systolic blood vessel diameter Ds1. The cardiac systolic blood pressure Ps1 and the cardiac diastolic blood pressure Pd1 are calculated from the correlation equation (1) between the blood vessel diameter D and the blood pressure P. Therefore, since the cardiac systolic blood pressure and the cardiac diastolic blood pressure appearing for each heartbeat can be measured before the next heartbeat arrives, it is possible to measure the highest blood pressure (cardiac systolic blood pressure) and the lowest blood pressure (cardiac diastolic blood pressure) quickly and continuously.

### Modification Examples

In addition, it should be understood that embodiments to which the invention can be applied are not limited to the embodiment described above and various modifications can be made without departing from the spirit and scope of the invention.

(A) In the embodiment described above, “acceleration at which the blood vessel diameter changes” is used as the “degree of change of the blood vessel diameter”, however “speed at which the blood vessel diameter changes” may also be used.

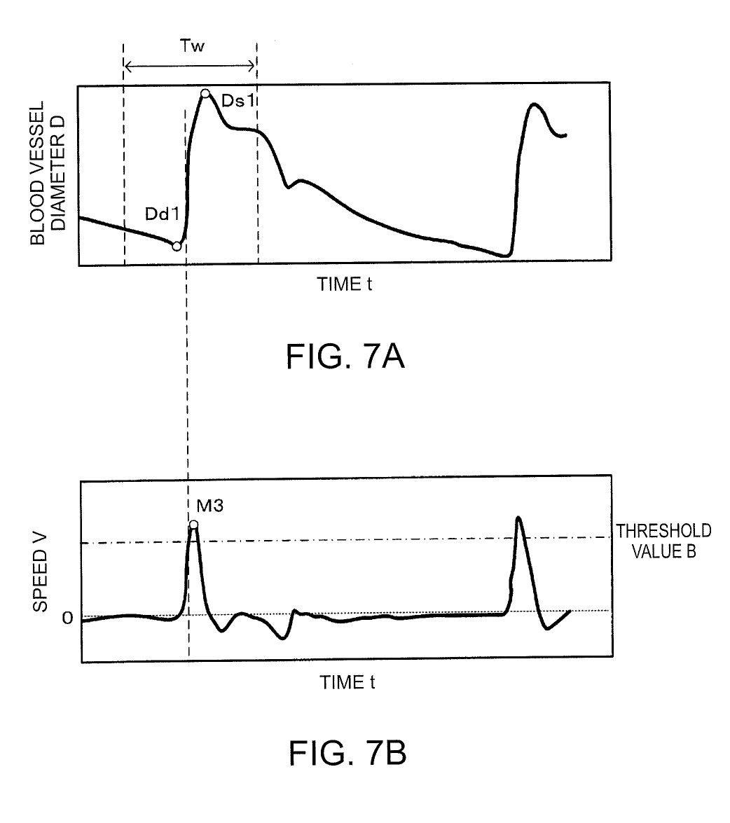

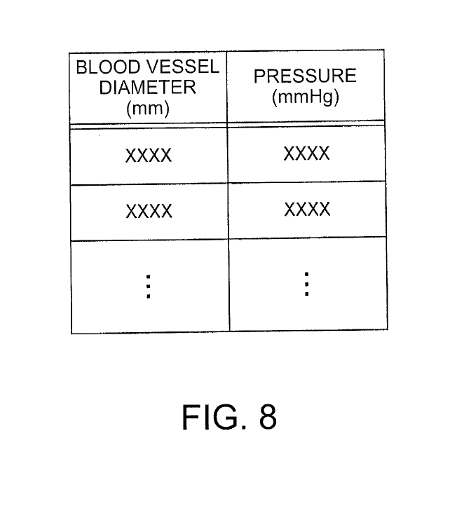

FIG. 7A is a blood vessel diameter variation waveform showing a temporal change in the blood vessel diameter of the carotid artery in one heartbeat period. FIG. 7B is a waveform showing a variation speed that is obtained from the blood vessel diameter variation waveform shown in FIG. 7A and is an example of the degree of change in the blood vessel diameter. The variation speed waveform of the blood vessel diameter is obtained by differentiating the blood vessel diameter waveform between adjacent frames once in a time direction based on the frame rate. For the variation speed, a speed in a direction in which the blood vessel diameter D increases is “positive”.

As shown in FIG. 7B, a positive peak M3 appears in cardiac systole of the variation speed waveform of the blood vessel diameter. In addition, although the negative peak appears in cardiac diastole, the negative peak is a small peak because the change in the blood vessel diameter is gentle compared with the cardiac systole. That is, the cardiac systolic blood vessel diameter Ds1 and the cardiac diastolic blood vessel diameter Dd1 appear before and after the peak M3 in cardiac systole of the variation speed waveform of the blood vessel diameter. Specifically, the peak M3 indicates a peak of the blood vessel diameter, the cardiac systolic blood vessel diameter Ds1 appears as a maximum value immediately after the peak M3, and the cardiac diastolic blood vessel diameter Dd1 appears as a minimum value immediately before the peak M3. Therefore, a predetermined period Tw including the peak M3 of the variation speed waveform of the blood vessel diameter can be set, and the maximum value of the blood vessel diameter D in the predetermined period Tw can be detected as the cardiac systolic blood vessel diameter Ds1 and the minimum value of the blood vessel diameter D can be detected as the cardiac diastolic blood vessel diameter Dd1.

In addition, the peak M3 in the variation speed waveform of the blood vessel diameter is detected by the variation speed exceeding a predetermined threshold value B, and a time at which the peak M3 is detected is determined to be the peak. The predetermined threshold value B is set to a value that is smaller than the peak M3 of cardiac systole in the variation speed waveform.

(B) In the embodiment described above, the communication method of the ultrasonic blood pressure measuring device 10 and the pressure sphygmomanometer 40 is wireless communication. However, wired communication using a cable may also be applied. In addition, the subject 2 may be made to measure the blood pressure using, for example, the pressure sphygmomanometer 40, and the subject 2 may manually input the measurement value to the ultrasonic blood pressure measuring device 10.

(C) In the embodiment described above, the blood vessel to be measured is the carotid artery. However, it is needless to say that other arteries, such as the radial artery, can be applied as an object to be measured.

(D) In addition, in the embodiment described above, the correlation setting data 330 is data for storing the value of each parameter defining the correlation equation (1). However, other forms may also be applied. For example, after deriving the correlation equation (1) by calculating the value of each parameter, a look-up table shown in FIG. 8 in which the correspondence relationship between the blood vessel diameter and blood pressure is set may be obtained from the correlation equation (1), and the look-up table may be set as the correlation setting data 330. A functional portion for obtaining the look-up table is a correlation setting section. The interval between blood vessel diameters in the look-up table can be arbitrarily set. For example, the interval between blood vessel diameters can be set to a unit of several to tens of micrometers.

In addition, the blood pressure calculation section 250 can calculate the cardiac systolic blood pressure Ps1 and the cardiac diastolic blood pressure Pd1 from the cardiac systolic blood vessel diameter Ds1 and the cardiac diastolic blood vessel diameter Dd1, which have been calculated by the blood vessel diameter feature value acquisition section 240, with reference to the look-up table. As a result, it is possible to reduce the calculation load when the blood pressure calculation section 250 calculates blood pressure.

What is claimed is:

1. An ultrasonic blood pressure measuring device comprising:
a blood vessel diameter measuring unit that measures a diameter of a blood vessel based on a reflected wave of an ultrasonic wave during at least one heartbeat period;
a peak calculation unit that calculates a peak of a degree of change in the blood vessel diameter;
a blood vessel diameter acquisition unit that (i) acquires a maximum value of the blood vessel diameter, which appears after the peak of the degree of change in the blood vessel diameter, as a cardiac systolic blood vessel diameter, and (ii) acquires a minimum value of the blood vessel diameter, which appears before the peak, as a cardiac diastolic blood vessel diameter; and
a blood pressure calculation unit that calculates cardiac systolic blood pressure and cardiac diastolic blood pressure from the cardiac systolic blood vessel diameter and the cardiac diastolic blood vessel diameter using a correlation between the diameter of the blood vessel and blood pressure set in advance.

2. The ultrasonic blood pressure measuring device according to claim 1,
wherein the blood vessel diameter acquisition unit acquires the cardiac systolic blood vessel diameter and the cardiac diastolic blood vessel diameter using a peak of a speed at which the blood vessel diameter changes as the peak of the degree of change.

3. The ultrasonic blood pressure measuring device according to claim 1,
wherein the blood vessel diameter acquisition unit acquires the cardiac systolic blood vessel diameter and the cardiac diastolic blood vessel diameter using a peak of acceleration at which the blood vessel diameter changes as the peak of the degree of change.

4. The ultrasonic blood pressure measuring device according to claim 1, further comprising:
a correlation setting unit that sets the correlation.

5. The ultrasonic blood pressure measuring device according to claim 1,
wherein the blood vessel is an artery, the ultrasonic blood pressure measuring device further comprising a transducer that transmits the ultrasonic wave to the artery, and receives the reflected wave of the ultrasonic wave from the artery.

6. The ultrasonic blood pressure measuring device according to claim 1, further comprising:
a storage unit that stores the correlation as a look-up table of the blood vessel diameter and the blood pressure,
wherein the blood pressure calculation unit calculates the cardiac systolic blood pressure and the cardiac diastolic blood pressure from the cardiac systolic blood vessel diameter and the cardiac diastolic blood vessel diameter with reference to the look-up table.

7. An ultrasonic blood pressure measuring method comprising:
receiving a reflected wave of an ultrasonic wave transmitted to a blood vessel and measuring a diameter of the blood vessel based on the reflected wave of the ultrasonic wave during at least one heartbeat period;
calculating a peak of a degree of change in the blood vessel diameter; and
calculating cardiac systolic blood pressure and cardiac diastolic blood pressure from (a) a maximum value of the blood vessel diameter, which appears after the peak of the degree of change in the blood vessel diameter, and (b) a minimum value of the blood vessel diameter, which appears before the peak, using a correlation between the diameter of the blood vessel and blood pressure set in advance.

8. The ultrasonic blood pressure measuring method according to claim 7,
Wherein the maximum value of the blood vessel diameter and the minimum value of the blood vessel diameter are acquired using a peak of a speed at which the blood vessel diameter changes as the peak of the degree of change.

9. The ultrasonic blood pressure measuring method according to claim 7,
Wherein the maximum value of the blood vessel diameter and the minimum value of the blood vessel diameter are acquired using a peak of acceleration at which the blood vessel diameter changes as the peak of the degree of change.

10. The ultrasonic blood pressure measuring method according to claim 7, further comprising:
storing the correlation between the diameter of the blood vessel and blood pressure set in advance as a look-up table of the blood vessel diameter and the blood pressure,
wherein the step of calculating the cardiac systolic blood pressure and the cardiac diastolic blood pressure from maximum value of the blood vessel diameter and the minimum value of the blood vessel diameter with reference to the look-up table.

11. An ultrasonic blood pressure measuring device comprising:
a transducer that transmits an ultrasonic wave to a blood vessel and receives a reflected wave of the ultrasonic wave; and
a hardware processor configured to:
measure a diameter of the blood vessel based on the reflected wave of the ultrasonic wave during at least one heartbeat period,
calculate a peak of a degree of change in the blood vessel diameter,
acquire a maximum value of the blood vessel diameter, which appears after the peak of the degree of change in the blood vessel diameter, as a cardiac systolic blood vessel diameter, and acquire a minimum value of the blood vessel diameter, which appears before the peak, as a cardiac diastolic blood vessel diameter, and
calculate cardiac systolic blood pressure and cardiac diastolic blood pressure from the cardiac systolic blood vessel diameter and the cardiac diastolic blood vessel diameter using a correlation between the diameter of the blood vessel and blood pressure set in advance.

12. The ultrasonic blood pressure measuring device according to claim 11,
wherein the hardware processor acquires the cardiac systolic blood vessel diameter and the cardiac diastolic blood vessel diameter using a peak of a speed at which the blood vessel diameter changes as the peak of the degree of change.

13. The ultrasonic blood pressure measuring device according to claim 11,
wherein the hardware processor acquires the cardiac systolic blood vessel diameter and the cardiac diastolic blood vessel diameter using a peak of acceleration at which the blood vessel diameter changes as the peak of the degree of change.

14. The ultrasonic blood pressure measuring device according to claim 11,
wherein the blood vessel is an artery, and the transducer is configured to transmit the ultrasonic wave to, and receive the reflected wave from, the artery.

15. The ultrasonic blood pressure measuring device according to claim 11, further comprising:
a memory that stores the correlation as a look-up table of the blood vessel diameter and the blood pressure,
wherein the hardware processor calculates the cardiac systolic blood pressure and the cardiac diastolic blood pressure from the cardiac systolic blood vessel diameter and the cardiac diastolic blood vessel diameter with reference to the look-up table.
